(19) United States
(12) Patent Application Publication
(10) Pub. No.: US 2013/0079893 A1
(43) Pub. Date: Mar. 28, 2013

(54) DEVICE AND METHOD FOR THE HEATING AND/OR TEMPERATURE CONTROL OF PROSTHESIS SOCKETS

(71) Applicant: Andre Marcel Emile Allemand, Corneilla la Riviere (FR)

(72) Inventor: Andre Marcel Emile Allemand, Corneilla la Riviere (FR)

(21) Appl. No.: 13/702,681

(22) PCT Filed: May 20, 2011
(86) PCT No.: PCT/FR11/51155

§ 371 Date: Dec. 7, 2012

(30) Foreign Application Priority Data
Jun. 7, 2010 (FR) 1054439

(51) Int. Cl. A61F 2/78

(52) U.S. Cl. CPC A61F 2/78
USPC 623/36

(57) ABSTRACT

A device for heating and/or controlling the temperature of the socket of an orthopaedic appliance, which receives the distal end of an amputation stump, the device including a heating element arranged in the prosthetic appliance such that the parts of the socket in contact with the distal end of the amputation stump are heated and/or maintained at a predetermined temperature. A method is also disclosed for improving the wearing comfort of an orthopaedic appliance, and to an orthopaedic appliance.

### TECHNICAL FIELD

The present invention relates to a device and a method for heating and/or controlling the temperature of the socket of an orthopaedic appliance for equipping an amputation stump.

The field of the invention is that of orthopaedic appliance and prostheses intended to amputees.

### STATE OF PRIOR ART

People with a missing limb frequently suffer from recurrent pains, also called phantom limb pain, which can be very annoying.

As described in the Article by H. Flor, N. Birbaumer and R. A. Sherman, “Phantom Limb Pain”, Pain: Clinical Updates, Volume VIII, n° 3, June 2000, these pains come from the fact that nerve terminations of the stump, that is the area in which ₜhe limb has been severed, remains sensitive to stimuli such as, for example, irritation and friction related to the stump activity in the prosthesis, and cold.

It is also shown in this article that these painful conditions are often related to a decrease in the surface blood flow rate on the distal part of the stumps and to the consequences caused on heat control of this part.

Indeed, the body physiological response to outside temperature variations normally consists in controlling the skin temperature, so as to maintain it in a thermal neutrality zone substantially between 30° C. and 33° C., by vasodilatation or vasoconstriction of the surface blood vessels, depending on whether it is an exposure to cold or heat.

In the case of the amputated area, when the vasodilatation mechanism is not working, the temperature is no longer properly controlled. It has been found experimentally that in the case of an exposure to cold, this surface temperature could be in the order of 4 to 5° C. lower than the temperature of the equivalent area (that is at the same position) of the healthy limb.

The injured nerve terminations are thus subjected to cold, which increases the discharge rate and triggers or strongly enhances pain sensations.

For many amputees, the pain can be relieved by heating the distal area of the amputation stump, for example in hot water. But this method requires removing any appliance and thus, is only usable at rest.

Heating patches are known, which are intended to be applied directly to the skin to locally heat a zone of the body. This solution is temporarily applicable to relieve pain, but only to the extent that the amputation stump is not inserted in an orthopaedic appliance such as a prosthesis, in which case friction and fulcrums would make it unbearable.

But the problem to avoid the stimulation of nerve terminations in the stump is particularly important within the scope of the appliance of the amputated limb with a prosthesis or any orthopaedic appliance. Indeed, the distal area of the amputated stump and scar tissues are directly in contact with elements of the socket. These elements generally consist of flexible silicone parts which avoid excessive mechanical stimulations of the stump, and parts of composite and/or metal materials for rigid portions. These prosthesis elements are subjected to the outside temperature and communicate it to the stump surface, with which they are in close contact, by heat conduction.

The object of the present invention is to provide a device that can be adapted to existing orthopaedic appliances, which can substantially improve the comfort thereof by avoiding a stimulation of nerve terminations of the stump under the effect of cold.

### DISCLOSURE OF THE INVENTION

This purpose is achieved with a device for heating and/or controlling the temperature of the socket of an orthopaedic appliance for receiving the distal end of an amputation stump, characterised in that it comprises a heating element arranged in said orthopaedic appliance such that the parts of said socket in contact with said distal end of the amputation stump are heated and/or maintained at a predetermined temperature.

In this manner, when the stump is inserted in its orthopaedic appliance, the sensitive parts comprising nerve terminations and scar tissues are maintained at a temperature enabling nerve stimulations to be minimized, and this regardless of the outside temperature.

Further, heating and/or controlling the temperature of the socket parts already intended to be in contact with the distal end of the stump has the advantage of avoiding to bring further elements in contact with the stump, which would be likely to cause additional tolerance problems (fulcrums, material, friction, . . . )

The heating element can be an element inserted in an orthopaedic appliance. This can also be a part of the socket which comprises heating means such as heating tracks, heating bodies or electrical resistors, in which case the heating element is included in a part of the orthopaedic appliance.

The heating element can be in the form of a flexible film, which film comprises a support of flexible substantially electrically insulating material and, on at least one surface of said support, tracks of substantially electrically conducting material capable of being heated under the effect of an electric current passing therethrough. These tracks can for example comprise a metal alloy having a relatively high resistivity such as Inconel® or cupronickel (alloy including copper and nickel) or even copper.

The heating element can be made for example in the form of a flexible printed circuit on which a network of tracks is etched or deposited according to a conventional electronics process such as masking, chemical etching or screen printing. This network of tracks can include a single or a plurality of tracks, distributed so as to cover at least one part of the surface of the heating element.

Under the action of the electric current passing therethrough, the network of tracks which can be distributed according to series and/or parallel electrical configurations is heated under the effect of ohmic losses, which generates the desired heat. The distribution in the tracks on the surface, as well as their local width, can be adjusted to generate particular temperature profiles at the surface of the heating element. For most applications, however, a substantially homogeneous distribution in the tracks on at least one part of the surface of the heating element is preferable.

The support of the heating element can include one of the following materials: polyimide, Kapton®, Teflon, silicone rubber, or any suitable flexible material.

Polyimide or Kapton® has in particular the advantage that it can be very thin while having a good mechanical strength. They allow to make heating elements which can be inserted between parts of the socket of an already existing orthopaedic appliance, or wherein this function has not been necessarily provided during design, without disturbing the operation thereof.

The heating element can further include a reflective metal layer, capable of reflecting a heat radiation, such as an aluminized sheet. This layer can preferably be provided on the heating element face opposite to the amputation stump. It enables heat transfers towards the stump to be improved while limiting heat losses in the orthopaedic appliance body.

Other layers of substantially thermally insulating materials, in any form, can of course be added to restrict heat losses at best.

The heating element can further comprise on (at least) one face thereof an adhesive layer, so as to be capable to be readily attached to an element of the socket. This adhesive layer can for example be provided on the face opposite the face supporting the reflective metal layer.

The heating element can comprise cutouts making it capable of being glued on a curved surface, while minimizing thicknesses and folds.

In particular, it can be of a substantially circular or elliptical shape, and comprises cutouts at radial orientations. It thus can be substantially in a flower shape with a central part and a plurality of “petals”, which makes it particularly suitable to gluing to

socket elements which closely fit the distal end of an amputation stump and which have a generally substantially hemispherical shape.

The device according to the invention can further comprise a temperature sensor arranged such as to measure the temperature in the vicinity of the heating element, and temperature controlling means capable of maintaining said heating element to a temperature setpoint.

The temperature sensor can be placed such as to measure a temperature representative of the temperature of the socket walls in contact with the stump.

The temperature sensor can comprise one of the following elements: platinum resistor, PT100, thermistor, thermocouple, or any other type of sensitive element. This element can be provided on the heating element. In particular, the temperature sensor can comprise an element having a very low thickness, such as a surface mounting component (SMC), welded to the heating element.

This embodiment enables a heating element which is in the form of a flexible printed circuit without any additional component in extra thickness to be obtained, which provides it with a mechanical strength and optimum integration possibilities. Further, the temperature sensor can be located close to the centre of the element with respect to the stump, which offers ideal measurement conditions.

The device according to the invention can further include at least one other temperature sensor for example measuring the room temperature, or the skin temperature in an area different from that of the stump.

The controlling means can include a single thermostat, which triggers or switches off the power supply to the heating element when the temperature measured exceeds a threshold. They can also include a microcontroller device which optimizes the power supply to the heating element as a function of a temperature measured and possibly other parameters such as outside temperature, skin temperature measured in an area different from the stump, service life or status of power supply batteries, . . . .

The device according to the invention can be designed so as to be able to maintain temperature setpoints in the order of 30 to 40° C. In

particular, the temperature setpoint can be substantially between 30° C. and 33° C., which corresponds to the “normal” temperature of the surface of the skin when physiological control mechanisms properly operate.

The device according to the invention can further include a case capable of being worn by the user and connected to the heating element by a connecting cable, which case comprises power supply batteries, and means for adjusting the temperature setpoint by the user.

The power supply batteries can for example be of the lithium-ion or lithium-polymer type.

The case can include fastening means such as a clip to be worn to a belt, or means to carry it as a shoulder bag or to fasten it to an orthopaedic appliance.

The connecting cable can include connection means for readily disconnecting the case, for example when the user changes his/her prosthesis (in which case the heating element remains in the prosthesis) or in the case of replacement of the heating element.

According to one embodiment, the heating element can be inserted between the flexible socket and the rigid socket of a limb prosthesis, and glued to said flexible socket.

According to another aspect of the invention, it is provided an orthopaedic appliance comprising a socket for receiving the distal end of an amputation stump, characterised in that it comprises a heating element arranged such that the parts of said socket in contact with said distal end of the amputation stump are heated and/or maintained at a predetermined temperature.

This orthopaedic appliance can be one of the following types: lower limb prosthesis for fitting a tibial amputation stump, lower limb prosthesis for fitting a femoral amputation stump, upper limb prosthesis.

According to yet another aspect of the invention, it is provided a method for improving the wearing comfort of an orthopaedic appliance comprising a socket for receiving the distal end of an amputation stump, characterised in that it comprises an operation of heating and/or maintaining parts of said socket in contact with said distal end of the amputation stump at a predetermined temperature, by means of a heating element arranged in said orthopaedic appliance.

The predetermined temperature can be substantially equal to the temperature of the skin surface in a healthy area of substantially equivalent anatomical location to that of the amputation stump, wherein the physiological mechanisms for controlling temperature properly operate.

### DESCRIPTION OF THE FIGURES AND EMBODIMENT

Further advantages and features of the invention will appear upon reading the detailed description of implementations and embodiments in no way limiting, and the following appended drawings:

FIG. 1 illustrates an embodiment of the device according to the invention,

FIG. 2 shows a top view of the heating element,

FIG. 3 illustrates a cross-section view of the heating element.

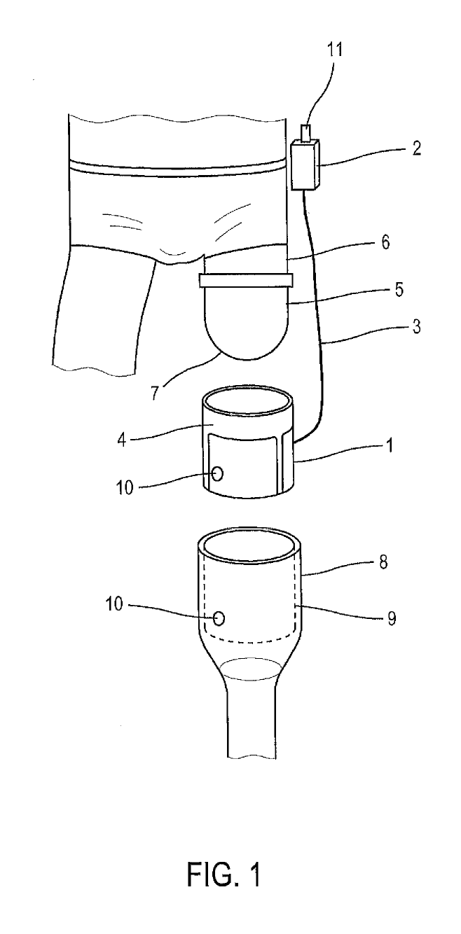

In reference to FIG. 1, an embodiment of the device according to the invention for equipping a femoral prosthesis will be described.

These prostheses are intended to fit amputation stumps 6 severed between the knee and the hip. The amputation stump 6 includes a distal end 7, which corresponds to the amputation place. In this distal end 7 are located a zone of scar tissues and injured nerve terminations as a result of amputation.

The distal end 7 of the amputation stump 6 is generally covered with a silicone sleeve 5 which covers and protects it.

According to an embodiment which is common to most limb prostheses, the femoral prosthesis includes a flexible socket 4, which is snap-fitted into the rigid socket 9 of the prosthesis 8 or by any other means. The sleeve 5 is substantially sealingly fitted into the flexible socket 4. A valve 10 enables air to be escaped from the flexible socket 4 upon placing the sleeve 5. This valve 10 is then closed back and the prosthesis 8 is maintained in place by the depression. To remove the prosthesis, to let air in through the valve 10 is sufficient.

The heating element 1 of the device according to the invention is made as a flexible film, which is glued around the base of the flexible socket 4. It is provided so as to substantially homogeneously heat the

part making up the base of the flexible socket 4 which is directly facing the distal end 7 of the amputation stump 6. In this manner, it is possible to heat or maintain at a determined temperature very homogeneously the entire distal area 7 comprising the sensitive scar part of the amputation stump 6.

The location of this heating element 1 in the socket is also selected so as not to disturb the operation of the socket parts such as the sleeve 5 and the flexible socket 4, which are in contact with the amputation stump 6 or in the immediate vicinity of the same, and which determine the comfort and the way the prosthesis 8 will be tolerated.

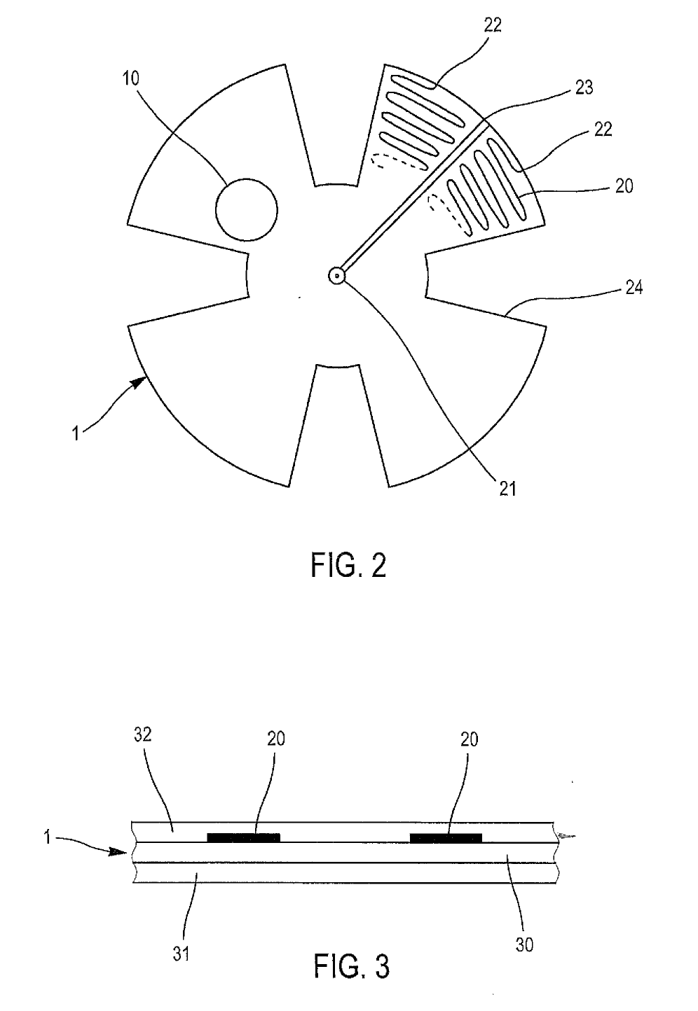

In reference to FIGS. 2 and 3, the heating element 1 is made based on a flexible printed circuit technology. It includes a support 30 of polyimide or Kapton®, wherein tracks 20 made of metal alloy such as Inconel® or cupronickel are etched. The material of the support 30 is selected depending on its flexibility and mechanical strength qualities. The tracks are achieved according to a conventional electronic process, for example etching.

The effective area of the heating element 1 is covered by a network of substantially uniformly distributed tracks 20. Only one part of this network is represented in FIG. 1 for the sake of legibility. The tracks are electrically connected to power supply means by connection points 22.

The network of tracks is calculated so as to have a resistance R in the order of 30 to 50 Ohm between connection points 22. Thus, when it is powered by a voltage U=11.1 volts, the heating element dissipates as heat a power P=̂2/R in the order of 3.5 watts.

The heating element 1 further includes a temperature sensor 21 facing its centre, at a position where the measured temperature is particularly representative to that the distal end 7 of the amputation stump 6 is subjected to. This temperature sensor 21 is connected to control means by connection points 23. The temperature sensor 21 is of the platinum resistor type (PT100), and has the form of a surface mounting component (SMC) having very low dimensions and above all, a very low height, lower than 0.5 mm.

In so far as the heating element 1 is intended to be inserted between socket parts of the prosthesis without disturbing the operation thereof while being subjected to potentially strong mechanism stresses, it is important that it should be made as a thin and resistant film, without remarkably projecting elements.

The heating element 1 includes cutouts 24. The shape of these cutouts 24 can of course be freely optimized for each application. The embodiment of FIG. 2 is suitable for the insertion into the femoral prosthesis of FIG. 1. The cutouts define petals which can be folded back and glued to a substantially hemispherical surface without excessive folding of the heating element 1.

The heating element 1 also includes an opening 10 for the passage of the valve which ensures sealing of the sleeve 5 fitting into the flexible socket 4.

FIG. 3 exhibits a cross-section view of the heating element 1. This representation is purely schematic and the relative thicknesses of drawn layers are not representative of the real dimensions. The total effective thickness of the heating element can be substantially lower than 1 mm, or even lower than 0.5 mm.

The heating element 1 comprises on faces thereof a glue adhesive layer 32. Thus, it can be readily placed by gluing on the flexible socket 4, and replaced as required.

The heating element 1 also includes a reflective layer 31, such as a thin aluminium film, glued onto the face directed to the rigid socket 9. The function of this reflective layer 31 is to improve the thermal efficiency by reflecting heat radiation substantially isotropically generated by the tracks 20 towards the stump 6.

The heating element 1 is connected by a connecting cable 3 and a connector to a control case 2 which can for example be attached to a user's belt by a clip. This case 2 includes power supply batteries, of the lithium-ion or lithium-polymer type for example, as well as the control and temperature control electronics, based on a microcontroller. It also includes a possibility for adjusting 11 temperature of the heating element 1 by the user.

The power of the heating element is calculated so as to allow for a rise in temperature of the distal part 7 of the amputation stump in the order of 4° C. at least in the environment of the prosthesis, so as to maintain this part in the thermal neutrality zone, that is about 30 to 33° C. Under these conditions, the capacity of the batteries affords an operating range of more than 6 hours.

The temperature adjustment enables the user to increase the temperature setpoint up to 40° C. at least for short durations, which enables him/her to lessen and relieve a breakthrough pain, or simply to choose a different comfort temperature.

According to embodiments:

the tracks or heating boy 20 can be made by any process, from which screen printing;

the heating element 1 can include a support 30 being moulded according to a particular shape, for example to fit the shape of a particular surface at best;

the heating element 1 can be of any shape, and include any type of cutouts, depending on the prosthesis it is intended to;

the heating element 1 can be integrated to an element of the prosthesis such as the flexible socket 4 or the rigid socket 9, or the body 8, during its manufacture, in particular by incorporating heating elements into this element.

Of course, the invention is not restricted to the examples just described and numerous alterations can be provided to these examples without departing from the scope of the invention.

1. A device for heating and/or controlling the temperature of the socket of an orthopaedic appliance for receiving a distal end of an amputation stump, comprising: a heating element arranged in the orthopaedic appliance such that parts of the socket in contact with the distal end of the amputation stump are heated and/or maintained at a predetermined temperature.

2. The device according to claim 1, characterised in that the heating element is in the form of a flexible film, which film comprises a support of flexible substantially electrically insulating material and, on at least one surface of said support, tracks of substantially electrically conducting material capable of being heated under the effect of an electric current passing therethrough.

3. The device according to claim 2, characterised in that the support comprises one of the following materials: polyimide, Kapton® film, silicone rubber.

4. The device according to claim 2, characterised in that the heating element further comprises a reflective metal layer, capable of reflecting a heat radiation.

5. The device according to claim 2, characterised in that the heating element further comprises an adhesive layer on one face thereof.

6. The device according to claim 2, characterised in that the heating element comprises cutouts making it capable of being glued on a curved surface.

7. The device according to claim 6, characterised in that the heating element is of substantially circular or elliptic shape, and comprises cutouts at radial orientations.

8. The device according to claim 2, characterised in that it further comprises a temperature sensor arranged so as to measure the temperature in the vicinity of the heating element, and temperature controlling means capable of maintaining said heating element to a temperature setpoint.

9. The device according to claim 8, characterised in that the temperature sensor comprises one of the following elements: platinum resistor, PT100, thermistor, thermocouple, arranged on the heating element.

10. The device according to claim 8, characterised in that the temperature setpoint is substantially between 30° C. and 33° C.

11. The device according to claim 8, characterised in that it further comprises a case capable of being worn by the user and connected to the heating element by a connecting cable, which case comprises power supply batteries, and means for adjusting the temperature setpoint by the user.

12. The device according to claim 2, characterised in that the heating element is inserted between the flexible socket and the rigid socket of a limb prosthesis, and glued to said flexible socket.

13. An orthopaedic appliance comprising: a socket for receiving the distal end of an amputation stump, further including a heating element arranged such that the parts of said socket in contact with the distal end of the amputation stump are heated and/or maintained at a predetermined temperature.

14. The orthopaedic appliance according to claim 13, characterised in that it is of one of the following types: lower limb prosthesis for fitting a tibial amputation stump, lower limb prosthesis for fitting a femoral amputation stump, upper limb prosthesis.

15. A method for improving the wearing comfort of an orthopaedic appliance including—a socket for receiving a distal end of an amputation stump, comprising: heating and/or maintaining parts of said socket in contact with the distal end of the amputation stump at a predetermined temperature, by means of a heating element arranged in said orthopaedic appliance.

16. The method according to claim 15, characterised in that the predetermined temperature is substantially equal to a temperature of the skin surface in a healthy area of substantially equivalent anatomical location to that of the amputation stump.
